(19) United States
(12) Patent Application Publication
(10) Pub. No.: US 2004/0043405 A1
(43) Pub. Date: Mar. 4, 2004

(54) Nucleic acid detection assay control genes

(75) Inventors: Arthur Castle, Gaithersburg, MD (US); Brandon Higgs, Gaithersburg, MD (US); Michael Elashoff, Gaithersburg, MD (US); Mark Porter, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: Gene Logic, Inc.

(21) Appl. No.: 10/620,765

(22) Filed: Jul. 17, 2003

Related U.S. Application Data
(60) Provisional application No. 60/396,145, filed on Jul. 17, 2002.

(51) Int. Cl. C12Q 1/68; G06F 19/00; G01N 33/48; G01N 33/50

(52) U.S. Cl. 435/6; 702/20

(57) ABSTRACT

The present invention includes methods of identifying genes whose expression level is invariant among cell or tissue types. The methods of the invention can be used in the diagnosis of disease, in quality control in evaluating external data or databases, and in normalization of external data for comparative purposes. The genes of the invention can be used to produce microarrays that generate data with improved reliability.

### RELATED APPLICATIONS

This application claims priority under 35 U.S.C. §119(e) to U.S. Provisional Application No. 60/396,145, filed Jul. 17, 2002, which is herein incorporated by reference in its entirety.

### FIELD OF THE INVENTION

The invention relates generally to control genes that may be utilized for normalizing hybridization and/or amplification reactions, as well as methods of identifying these genes that may be used in toxicology studies and in analyzing gene expression data sets for quality and compatibility with other data sets.

### BACKGROUND OF THE INVENTION

Nucleic acid hybridization and other quantitative nucleic acid detection assays are routinely used in medical and biotechnological research and development, diagnostic testing, drug development and forensics. Such technologies have been used to identify genes which are up- or down-regulated in various disease or physiological states, to analyze the roles of the members of cellular signaling cascades and to identify drugable targets for various disease and pathology states.

Examples of technologies commonly used for the detection and/or quantification of nucleic acids include Northern blotting (Krumlauf (1994), Mol Biotechnol 2:227-242), in situ hybridization (Parker & Barnes (1999), Methods Mol Biol 106:247-283), RNAse protection assays (Hod (1992), Biotechniques 13:852-854; Saccomanno et al. (1992), Biotechniques 13:846-850), microarrays, and reverse transcription polymerase chain reaction (RT-PCR) (see Bustin (2000), J Mol Endocrin 25:169-193).

The reliability of these nucleic acid detection methods depend on the availability of accurate means for accounting for variations between analyses. For example, variations in hybridization conditions, label intensity, reading and detector efficiency, sample concentration and quality, background effects, and image processing effects each contribute to signal heterogeneity (Hegde et al. (2000), Biotechniques 29:548-562; Berger et al. (2000), WO 00/04188). Normalization procedures used to overcome these variations often rely on control hybridizations to housekeeping genes such as β-actin, glyceraldehyde-3-phosphate dehydrogenase (GADPH), and the transferrin receptor gene (Eickhoff et al. (1999), Nuc Acids Res 27:e33; Spiess et al. (1999), Biotechniques 26: 46-50). These methods, however, generally do not provide the signal linearity sufficient to detect small but significant changes in transcription or gene expression (Spiess et al. (1999), Biotechniques 26: 46-50). In addition, the steady state levels of many housekeeping genes are susceptible to alterations in expression levels that are dependent on cell differentiation, nutritional state, specific experimental and stimulation protocols (Eickhoff et al. (1999), Nuc Acids Res 27:e33; Spiess et al. (1999), Biotechniques 26:46-50; Hegde et al. (2000), Biotechniques 29:548-562; and Berger et al. (2000), WO 00/04188). Consequently, there exists a need for the identification and use of additional genes that may serve as effective controls in nucleic acid detection assays.

### SUMMARY OF THE INVENTION

The present invention includes methods of identifying at least one gene that is consistently or invariantly expressed across different cell or tissue types in an organism, comprising: preparing gene expression profiles for different cell or tissue types from the organism; calculating a percent variability of expression for at least one gene in each of the profiles across the different cell or tissue types; and selecting any gene whose percent variability of expression indicates that the gene is consistently or invariantly expressed across the different cell or tissue types. The percent variability of expression may be determined by a one-factor or two-factor analysis of variance (ANOVA) wherein the R² value is a measure of percent variability of expression.

The invention, in another embodiment, includes methods of normalizing the data from a nucleic acid detection assay comprising: detecting the expression level for at least one gene in a nucleic acid sample; and normalizing the expression of said at least one gene with the detected expression of at least one control gene of Table 1. The number of control genes used to normalize gene expression data may comprise about 10, 25, 50, 100, 500 or more of the control genes herein identified.

In another embodiment, the invention includes a set of probes comprising at least two probes that specifically hybridize to a gene of Table 1. The set may comprise at least about 10, 25, 50, 100, 500 or more of the control genes of Table 1. The sets of probes may or may not be attached to a solid substrate such as a chip.

### DETAILED DESCRIPTION

The present Inventors have identified rat control genes that may be monitored in nucleic acid detection assays and whose expression levels may be used to normalize gene expression data or evaluate the suitability of test data to compare to or to include in a database of like data. Normalization of gene expression data from a cell or tissue sample with the expression level(s) of the identified control genes allows the accurate assessment of the expression level(s) for genes that are differentially regulated between samples, tissues, treatment conditions, etc. These control genes may be used across a broad spectrum of assay formats, but are particularly useful in microarray or hybridization based assay formats.

A. Nucleic Acid Detection Assay Controls

1. Selection of Control Genes

As used herein, the genes selected by the disclosed methods as well as the rat genes and nucleic acids of Table 1 (identified by ANOVA methods, discussed below) are referred to as “invariant” or “control genes.” Control genes of the invention may be produced by a method comprising preparing gene expression profiles (a representation of the expression level for at least one gene, preferably 10, 25, 50, 100, 500 or more, or, most preferably, nearly all or all expressed genes in a sample) from at least two (or a variety) of cell or tissue types, or from a set of samples of at least one cell or tissue type in which the set contains normal samples (from healthy animals), disease state samples, toxin-exposed samples, etc., measuring the level of expression for at least one gene in each of the gene expression profiles to produce gene expression data, calculating the variation in expression level (R²) from the gene expression data for each gene and selecting genes whose variation in expression level indicates that the gene is consistently expressed at about the same level in the different cell or tissue types. In one embodiment, such genes that are expressed at about the same level, or are invariantly expressed, are those genes that have a percent variability in expression level (R²) less than or equal to about 12.

In preferred embodiments, the statistical measure referred to herein as the percent variability in expression level (R²) is calculated on a gene by gene basis across a number of samples or across a reference database to find the least variant genes with respect to a number of cell or tissue types or sample treatments. A two-factor ANOVA model is applied to all cell and tissue sample sets where both control and disease, pathology or treatment groups exist. The factors for this model were normal state (control or affected tissue) and tissue type. A one factor ANOVA was also used to examine the effects of tissue kind alone. Genes are ranked according to R-squared values. The R-squared value can be interpreted as the percent variability of expression that can be explained by the underlying factors. Cut-off values are also selected for the alpha error p-values for each factor and the interaction of these two factors. A cut-off value for both one factor and two factor R² values of less than or equal to about 14, preferably less than about 12, may be used, and genes with R² values less than or equal to 14, preferably less than or equal to 12, may be selected as control genes or considered as genes that are consistently expressed across the different cell or tissue types tested. In addition, any gene with large known regulation events within tissues may be removed and any co-clustered Unigene fragments may be examined for consistency in R² values. A probe set is also selected using the following supplemental criteria: (a) Mean Average Differential over all rat samples less than or equal to about 20, (b) Present Frequency over all rat samples less than or equal to about 75% and (c) no probe sets exhibiting saturation.

Eᵢⱼ=u+Tⱼ+error Model 1

(Eᵢⱼ is the expression value of the i^{th} gene in the j^{th} sample)

(Tⱼ is the tissue type of the j^{th} sample)

For each gene, model fitting produces a p-value for the T factor, as well as a sum of squares attributable to this factor. This sum of squares is the model sum of squares. The R² value is then the ratio of the model sum of squares to the total sum of squares

$${\sum }_{j}^{} {( {E}_{ij} - {\overline{E}}_{i} )}^{2} .$$

Eᵢⱼ=u+Tⱼ+Nⱼ+Tⱼ*Nⱼ+error Model 2

(Eᵢⱼ is the expression value of the i^{th} gene in the j^{th} sample)

(Tⱼ is the tissue type of the j^{th} sample)

(Nⱼ is the state of the j^{th} sample (Nⱼ=0 for normal, 1 otherwise))

The model fitting yields, for each gene, a p-value for the T factor, the N factor, and the T*N factor, as well as a sum of squares attributable to each of these factors. Adding the three sums of squares gives the model sum of squares. The R² value is then the ratio of the model sum of squares to the total sum of squares

$${\sum }_{j}^{} {( {E}_{ij} - {\overline{E}}_{i} )}^{2} .$$

Further, the ANOVA-based methods of the invention are particularly useful for determining the compatibility of a test sample to an entire set of samples, or an existing database derived from those samples. For instance, an R² value for genes that have been shown to be the most resistant to variability is calculated for all samples within a test group or test database. These R² values are then compared to those from a standard reference database. Accordingly, a closeness distribution of all individual samples in the test database to the reference database as a whole can be generated to evaluate the compatibility of new samples. The genes identified in Table 1 show invariant patterns of expression and can be used to assess compatibility and reliability of gene expression experiments and predictive modeling experiments. These genes show low variability both in control groups from many different experiments and in studies of disruptions of gene expression, such as those occurring in disease states. As a result, these genes can be used as an internal standard for comparing gene expression data. Measurements of expression level of these genes are used to determine the extent of compatibility of data from different sources and the need, or lack thereof, for normalization or further quality control and adjustments. These measurements also provide an internal standard that supplies a reference point for highly disrupted patterns of gene expression. These genes are also of critical importance for determining relative expression if small numbers of markers are used in custom microarrays.

In some embodiments of the invention, the percent variability of expression may be calculated from data that has been normalized to control for the mechanics of hybridization, such as data normalized or controlled for background noise due to non-specific hybridization. Such data typically include, but are not limited to, fluorescence readings from microarray based hybridizations, densitometry readings produced from assays that rely on radiological labels to detect and quantify gene expression and data produced from quantitative or semi-quantitative amplification assays.

In the methods of the invention, gene expression profiles may be produced by any means of quantifying gene expression for at least one gene in the tissue or cell sample. In preferred methods, gene expression is quantified by a method selected from the group consisting of a hybridization assay or an amplification assay. Hybridization assays may be any assay format that relies on the hybridization of a probe or primer to a nucleic acid molecule in the sample. Such formats include, but are not limited to, differential display formats and microarray hybridization, including microarrays produced in chip format. Amplification assays include, but are not limited to, quantitative PCR, semiquantitative PCR and assays that rely on amplification of nucleic acids subsequent to the hybridization of the nucleic acid to a probe or primer. Such assays include the amplification of nucleic acid molecules from a sample that are bound to a microarray or chip.

In other circumstances, gene expression profiles may be produced by querying a gene expression database comprising expression results for genes from various cell or tissue samples. The gene expression results in the database may be produced by any available method, such as differential display methods and microarray-based hybridization methods. The gene expression profile is typically produced by the step of querying the database with the identity of a specific cell or tissue type for the genes that are expressed in the cell or tissue type and/or the genes that are differentially regulated compared to a control cell or tissue sample. Available databases include, but are not limited to, the Gene Logic ToxExpress® database, the Gene Expression Omnibus gene expression and hybridization array repository available through NCBI (www.ncbi.nlm.nih.gov/entrez) and the SAGE™ gene expression database.

The cell or tissue samples that are used to prepare gene expression profiles may include any cell or tissue sample available. Such samples include, but are not limited to, tissues removed as surgical samples, diseased or normal tissues, in vitro or in vivo grown cells, and cell cultures and cells or tissues from animals exposed to an agent such as a toxin. The number of samples that may be used to calculate absolute R² values is variable, but may include about 3, 10, 25, 50, 100, 200, 500 or more cell or tissue samples. The cell or tissue samples may be derived from an animal or plant, preferably a mammal, most preferably a rat. In some instances, the cell or tissue samples may be human, canine (dog), or mouse in origin.

As used herein, “background” refers to signals associated with non-specific binding (cross-hybridization). In addition to cross-hybridization, background may also be produced by intrinsic fluorescence of the hybridization format components themselves.

“Bind(s) substantially” refers to complementary hybridization between an oligonucleotide probe and a nucleic acid sample and embraces minor mismatches that can be accommodated by reducing the stringency of the hybridization media to achieve the desired detection of the nucleic acid sample.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

2. Preparation of Controls Genes, Probes and Primers

The control genes listed in Table 1 may be obtained from a variety of natural sources such as organisms, organs, tissues and cells. The sequences of known genes are in the public databases. The GenBank Accession Number corresponding to the Normalization Control Genes can be found in Table 1. The sequences of the genes in GenBank (http://www.ncbi.nlm.nih.gov/) are herein incorporated by reference in their entirety as of the priority date of this application.

Probes or primers for the nucleic acid detection assays described herein that specifically hybridize to a control gene may be produced by any available means. For instance, probe sequences may be prepared by cleaving DNA molecules produced by standard procedures with commercially available restriction endonucleases or other cleaving agents. Following isolation and purification, these resultant normalization control gene fragments can be used directly, amplified by PCR methods or amplified by replication on or expression from a vector.

Control genes and control gene probes or primers (i.e., synthetic oligonucleotides and polynucleotides) are most easily synthesized by chemical techniques, for example, the phosphoramidite method of Matteucci, et al. ((1981) J Am Chem Soc 103:3185-3191) or using automated synthesis methods using the GenBank sequences disclosed in Table 1. Probes for attachment to microarrays or for use as primers in amplification assays may be produced from the sequences of the genes identified herein using any available software, including, for instance, software available from Molecular Biology Insights, Olympus Optical Co. and Premier Biosoft International.

In addition, larger nucleic acids can readily be prepared by well known methods, such as synthesis of a group of oligonucleotides that define various modular segments of the normalization control genes and normalization control gene segments, followed by ligation of oligonucleotides to build the complete nucleic acid molecule.

B. Normalization Methods

Gene expression data produced from the control genes in a given sample or samples may be used to normalize the gene expression data from other genes using any available arithmatic or calculative means. In particular, gene expression data from the control genes in Table 1 are useful to normalize gene expression data for toxicology testing or modeling in an animal model, preferably in a rat. Such methods include, but are not limited, methods of data analysis described by Hegde et al. (2000), Biotechniques 29:548-562; Winzeller et al. (1999), Meth Enzymol 306:3-18; Tkatchenko et al. (2000), Biochimica et Biophysica Acta 1500:17-30; Berger et al. (2000), WO 00/04188; Schuchhardt et al. (2000), Nuc Acids Res 28:e47; Eickhoff et al. (1999), Nuc Acids Res 27:e33. Micro-array data analysis and image processing software packages and protocols, including normalization methods, are also available from BioDiscovery (http://www.biodiscovery.com), Silicon Graphics (http://www.sigenetics.com), Spotfire (http://www.spotfire.com), Stanford University (http://rana.Stanford.EDU/software), National Human Genome Research Institute (http://www.nhgri.nih.gov/DIR/LCG/15K/HTML/img_analysis.html), TIGR (http://www.tigr.org/softlab), and Affymetrix (affy and maffy packages), among others.

C. Assay or Hybridization Formats

The control genes of the present invention may be used in any nucleic acid detection assay format, including solution-based and solid support-based assay formats. As used herein, “hybridization assay format(s)” refer to the organization of the oligonucleotide probes relative to the nucleic acid sample. The hybridization assay formats that may be used with the control genes and methods of the present invention include assays where the nucleic acid sample is labeled with one or more detectable labels, assays where the probes are labeled with one or more detectable labels, and assays where the sample or the probes are immobilized. Hybridization assay formats include but are not limited to: Northern blots, Southern blots, dot blots, solution-based assays, branched-DNA assays, PCR, RT-PCR, quantitative or semi-quantitative RT-PCR, microarrays and biochips.

As used herein, “nucleic acid hybridization” simply involves contacting a probe and nucleic acid sample under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label.

It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization, and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

As used herein, the term “stringent conditions” refers to conditions under which a probe will hybridize to a complementary control nucleic acid, but with only insubstantial hybridization to other sequences. Stringent conditions are sequence-dependent and will be different under different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above that the hybridization pattern is not appreciably altered and which provides adequate signal for the particular oligonucleotide probes of interest.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical residue (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights. Sequences corresponding to the control genes of Table 1 may comprise at least about 70% sequence identity to the GenBank IDs of the genes in the Tables, preferably about 75%, 80% or 85% or more preferably, about 90% or 95% or more identity.

Homology or identity is determined by BLAST (Basic Local Alignment Search Tool) analysis using the algorithm employed by the programs blastp, blastn, blastx, tblastn and tblastx (Karlin et al. (1990), Proc Natl Acad Sci USA 87:2264-2268 and Altschul (1993), J Mol Evol 36:290-300, fully incorporated by reference) which are tailored for sequence similarity searching. The approach used by the BLAST program is first to consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994), Nat Genet 6:119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al. (1992), Proc Natl Acad Sci USA 89:10915-10919, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

As used herein a “probe” or “oligonucleotide probe” is defined as a nucleic acid, capable of binding to a nucleic acid sample or complementary control gene nucleic acid through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Probe arrays may contain at least two or more oligonucleotides that are complementary to or hybridize to one or more of the control genes described herein. Such arrays may also contain oligonucleotides that are complementary or hybridize to at least about 2, 3, 5, 7, 10, 50, 100 or more the genes described herein. Any solid surface to which oligonucleotides or nucleic acid sample can be bound, either directly or indirectly, either covalently or non-covalently, can be used. For example, solid supports for various hybridization assay formats can be filters, polyvinyl chloride dishes, silicon or glass based chips, etc. Glass-based solid supports, for example, are widely available, as well as associated hybridization protocols. (see, e.g., Beattie, WO 95/11755).

A preferred solid support is a high density array or DNA chip. This contains an oligonucleotide probe of a particular nucleotide sequence at a particular location on the array. Each particular location may contain more than one molecule of the probe, but each molecule within the particular location has an identical sequence. Such particular locations are termed features. There may be, for example, 2, 10, 100, 1000, 10,000, 100,000, 400,000, 1,000,000 or more such features on a single solid support. The solid support, or more specifically, the area wherein the probes are attached, may be on the order of a square centimeter.

1. Dot Blots

The control genes listed in Table 1 and methods of the present invention may be utilized in numerous hybridization formats such as dot blots, dipstick, branched DNA sandwich and ELISA assays. Dot blot hybridization assays provide a convenient and efficient method of rapidly analyzing nucleic acid samples in a sensitive manner. Dot blots are generally as sensitive as enzyme-linked immunoassays. Dot blot hybridization analyses are well known in the art and detailed methods of conducting and optimizing these assays are detailed in U.S. Pat. Nos. 6,130,042 and 6,129,828, and Tkatchenko et al. (2000), Biochimica et Biophysica Acta 1500:17-30. Specifically, a labeled or unlabeled nucleic acid sample is denatured, bound to a membrane (i.e., nitrocellulose) and then contacted with unlabeled or labeled oligonucleotide probes. Buffer and temperature conditions can be adjusted to vary the degree of identity between the oligonucleotide probes and nucleic acid sample necessary for hybridization.

Several modifications of the basic Dot blot hybridization format have been devised. For example, Reverse Dot blot analyses employ the same strategy as the Dot blot method, except that the oligonucleotide probes are bound to the membrane and the nucleic acid sample is applied and hybridized to the bound probes. Similarly, the Dot blot hybridization format can be modified to include formats where either the nucleic acid sample or the oligonucleotide probe is applied to microtiter plates, microbeads or other solid substrates.

2. Membrane-Based Formats

Although each membrane-based format is essentially a variation of the Dot blot hybridization format, several types of these formats are preferred. Specifically, the methods of the present invention may be used in Northern and Southern blot hybridization assays. Although the methods of the present invention are generally used in quantitative nucleic acid hybridization assays, these methods may be used in qualitative or semiquantitative assays such as Southern blots, in order to facilitate comparison of blots. Southern blot hybridization, for example, involves cleavage of either genomic or cDNA with restriction endonucleases followed by separation of the resultant fragments on a polyacrylamide or agarose gel and transfer of the nucleic acid fragments to a membrane filter. Labeled oligonucleotide probes are then hybridized to the membrane-bound nucleic acid fragments. In addition, intact cDNA molecules may also be used, separated by electrophoresis, transferred to a membrane and analyzed by hybridization to labeled probes. Northern analyses, similarly, are conducted on nucleic acids, either intact or fragmented, that are bound to a membrane. The nucleic acids in Northern analyses, however, are generally RNA.

3. Arrays

Any microarray platform or technology may be used to produce gene expression data that may be normalized with the control genes and methods of the invention. Oligonucleotide probe arrays can be made and used according to any techniques known in the art (see for example, Lockhart et al., (1996), Nat Biotechnol 14:1675-1680; McGall et al. (1996), Proc Natl Acad Sci USA 93:13555-13460). Such probe arrays may contain at least one or more oligonucleotides that are complementary to or hybridize to one or more of the nucleic acids of the nucleic acid sample and/or the control genes of Tables 1-3. Such arrays may also contain oligonucleotides that are complementary or hybridize to at least 2, 3, 5, 7, 10, 25, 50, 100, 500 or more of the control genes listed in Tables 1-3.

Control oligonucleotide probes of the invention are preferably of sufficient length to specifically hybridize only to appropriate, complementary genes or transcripts. Typically the oligonucleotide probes will be at least about 10, 12, 14, 16, 18, 20 or 25 nucleotides in length. In some cases longer probes of at least 30, 40, or 50 nucleotides will be desirable. The oligonucleotide probes of high density array chips include oligonucleotides that range from about 5 to about 45 or 5 to about 500 nucleotides, more preferably from about 10 to about 40 nucleotides and most preferably from about 15 to about 40 nucleotides in length. In other particularly preferred embodiments, the probes are 20 or 25 nucleotides in length. In another preferred embodiment, probes are double- or single-stranded DNA sequences. The oligonucleotide probes are capable of specifically hybridizing to the control gene nucleic acids in a sample.

One of skill in the art will appreciate that an enormous number of array designs comprising control probes of the invention are suitable for the practice of this invention. The high density array will typically include a number of probes that specifically hybridize to each control gene nucleic acid, e.g. mRNA or cRNA. (See WO 99/32660 for methods of producing probes for a given gene or genes). Assays and methods comprising control probes of the invention may utilize available formats to simultaneously screen at least about 100, preferably about 1000, more preferably about 10,000 and most preferably about 500,000 or 1,000,000 different nucleic acid hybridizations.

The methods and control genes of this invention may also be used to normalize gene expression data produced using commercially available oligonucleotide arrays that contain or are modified to contain control gene probes or the invention. A preferred oligonucleotide array may be selected from the Affymetrix, Inc. GeneChip® series of arrays which include the Human Genome Focus Array, Human Genome U133 Set, Human Genome U95 Set, HuGeneFL Array, Human Cancer Array, HuSNP Mapping Array, GenFlex Tag Array, p53 Assay Array, CYP450 Assay Array, Rat Genome U34 Set, Rat Neurobiology U34 Array, Rat Toxicology U34 Array, Murine Genome U74v2 Set, Murine 11K Set, Yeast Genome S98 Array, E. coli Antisense Genome Array, E. coli Genome Array (Sense), Arabidopsis ATH1 Genome Array, Arabidopsis Genome Array, Drosophila Genome Array, C. elegans Genome Array, P. aeruginosa Genome Array and B. subtilis Genome Array. In another embodiment, an oligonucleotide array may be selected from the Motorola Life Sciences and Amersham Pharmaceuticals CodeLink™ Bioarray System microarrays, including the UniSet Human 20K I, Uniset Human I, ADME-Rat, UniSet Rat I and UniSet Mouse I, or from the Motorola Life Sciences eSensor™ series of microarrays.

4. RT-PCR

The control genes and methods of the invention may be used in any type of polymerase chain reaction. A preferred PCR format is reverse transciptase polymerase chain reaction (RT-PCR), an in vitro method for enzymatically amplifying defined sequences of RNA (Rappolee et al. (1988), Science 241:708-712) permitting the analysis of different samples from as little as one cell in the same experiment (See Ambion: RT-PCR: The Basics; M. J. McPherson and S. G. Møller, PCR BIOS Scientific Publishers Ltd., Oxford, OX4 1RE, 2000; Dieffenbach et al., PCR Primer: A Laboratory Manual, Cold Spring Harbor Laboratory Press, Cold Spring Harbor, N.Y., 1995, for review). One of ordinary skill in the art may appreciate the enormous number of variations in RT-PCR platforms that are suitable for the practice of the invention, including complex variations aimed at increasing sensitivity such as semi-nested (Wasserman et al. (1999), Mol Diag 4:21-28), nested (Israeli et al. (1994), Cancer Res 54:6303-6310; Soeth et al. (1996), Int J Cancer 69:278-282), and even three-step nested (Funaki et al. (1997), Life Sci 60:643-652; Funaki et al. (1998), Brit J Cancer 77:1327-1332).

In one embodiment of the invention, separate enzymes are used for reverse transcription and PCR amplification. Two commonly used reverse transcriptases, for example, are avian myeloblastosis virus and Moloney murine leukaemia virus. For amplification, a number of thermostable DNA-dependent DNA polymerases are currently available, although they differ in processivity, fidelity, thermal stability and ability to read modified triphosphates such as deoxyuridine and deoxyinosine in the template strand (Adams et al. (1994), Bioorg Med Chem 2:659-667; Perler et al. (1996), Adv Prot Chem 48:377-435). The most commonly used enzyme, Taq DNA polymerase, has a 5′-3′ nuclease activity but lacks a 3′-5′ proofreading exonuclease activity. When fidelity is required, proofreading exonucleases such as Vent and Deep Vent (New England Biolabs) or Pfu (Stratagene) may be used (Cline et al. (1996), Nuc Acids Res 24:3456-3551). In another embodiment of the invention, a single enzyme approach may be used involving a DNA polymerase with intrinsic reverse transcriptase activity, such as Thermus thermophilus (Tth) polymerase (Bustin (2000), J Mol Endo 25:169-193). A skilled artisan may appreciate the variety of enzymes available for use in the present invention.

The methodologies and control gene primers of the present invention may be used, for example, in any kinetic RT-PCR methodology, including those that combine fluorescence techniques with instrumentation capable of combining amplification, detection and quantification (Orlando et al. (1998), Clin Chem Lab Med 36:255-269). The choice of instrumentation is particularly important in multiplex RT-PCR, wherein multiple primer sets are used to amplify multiple specific targets simultaneously. This requires simultaneous detection of multiple fluorescent dyes. Accurate quantitation while maintaining a broad dynamic range of sensitivity across mRNA levels is the focus of upcoming technologies, any of which are applicable for use in the present invention. Preferred instrumentation may be selected from the ABI Prism 7700 (Perkin-Elmer-Applied Biosystems), the Lightcycler (Roche Molecular Biochemicals) and iCycler Thermal Cycler. Featured aspects of these products include high-throughput capacities or unique photodetection devices.

Without further description, it is believed that one of ordinary skill in the art can, using the preceding description and the following illustrative examples, practice the methods and use the control genes of the present invention. The following examples therefore, specifically point out the preferred embodiments of the present invention, and are not to be construed as limiting in any way the remainder of the disclosure.

### EXAMPLES

### Example 1

### Selection of Control Genes

The control genes were selected by querying a Gene Logic rat tissue database to create expression profiles from a variety of rat cell and tissue samples.

This database was produced from data derived from screening various cell or tissue samples using the Affymetrix rat GeneChip® set. The rat cell and tissue samples that were analyzed include those that were not treated at all and can be referred to as “normal,” as they represent the laboratory rat population that has not been manipulated outside of normal daily activity within that setting. In general, tissue and cell samples were processed following the Affymetrix GeneChip® Expression Analysis Manual. Frozen cells were ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg cells. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 3.0 and Expression Data Mining Tool (EDMT) software (version 1.0), S-Plus, and the GeneExpress® software system. Microarrays were scanned on a high photomultiplier tube (PMT) settings.

To prepare tissue samples from animals, e.g. rats, sterile instruments were used to sacrifice the animals, and fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissues were collected and processed as follows.

Liver

1. Right medial lobe—snap frozen in liquid nitrogen and stored at ˜−80° C.

2. Left medial lobe—Preserved in 10% neutral-buffered formalin (NBF) and evaluated for gross and microscopic pathology.

3. Left lateral lobe—snap frozen in liquid nitrogen and stored at ˜−80° C.

Heart—A sagittal cross-section containing portions of the two atria and of the two ventricles was preserved in 10% NBF. The remaining heart was frozen in liquid nitrogen and stored at ˜−80° C.

Kidneys (Both)

1. Left—Hemi-dissected; half was preserved in 10% NBF and the remaining half was frozen in liquid nitrogen and stored at ˜−80° C.

2. Right—Hemi-dissected; half was preserved in 10% NBF and the remaining half was frozen in liquid nitrogen and stored at ˜−80° C.

Testes (both)—A sagittal cross-section of each testis was preserved in 10% NBF. The remaining testes were frozen together in liquid nitrogen and stored at ˜−80° C.

Brain (whole)—A cross-section of the cerebral hemispheres and of the diencephalon was preserved in 10% NBF, and the rest of the brain was frozen in liquid nitrogen and stored at ˜−80° C.

Gene expression data were then analyzed to identify those genes that were consistently expressed across a set of about 5,000 different tissue samples. Table 1 provides a list of approximately 128 genes whose expression, as determined by ANOVA, is considered not to vary across the normal and treated samples studied. Table 1 also provides a GenBank Accession number (fragment name), present frequency and mean average differential for each of the genes. The GenBank Accession Nos. can be used to locate the publicly available sequences, each of which is herein incorporated by reference as of the priority date of this application (Jul. 17, 2002).

A two-factor ANOVA model was applied to all cell and tissues samples where both control and disease, pathology or treatment groups existed. The factors for this model were normal state (control or affected tissue) and cell or tissue type. A one factor ANOVA was also used to examine the effects of tissue kind alone. Genes were ranked according to R-squared values. The R-squared value can be interpreted as the percent variability of expression that can be explained by the underlying factors. Cut-off values were also selected for the alpha error p-values for each factor and the interaction of these two factors. A cut-off value for both one factor and two factor R-squared values of less than or equal to 12 was used. In addition, any gene with large known regulation events within tissues was removed and any co-clustered Unigene fragments were examined for consistency in R-Squared values. The probe set was also selected using the following supplemental criteria: (a) Mean Average Differential over all rat samples less than or equal to about 20, (b) Present Frequency over all rat samples less than or equal to about 75% and (c) no probe sets exhibiting saturation.

Eᵢⱼ=u+Tⱼ+error Model 1

(Eᵢⱼ is the expression value of the i^{th} gene in the j^{th} sample)

(Tⱼ is the tissue type of the j^{th} sample)

The model fitting yields, for each gene, a p-value for the T factor, as well as a sum of squares attributable to this factor. This sum of squares is the model sum of squares. The R² value is then the ratio of the model sum of squares to the total sum of squares

$${\sum }_{j}^{} {( {E}_{ij} - {\overline{E}}_{i} )}^{2} .$$

Eᵢⱼ=u+Tⱼ+Nⱼ+Tⱼ*Nⱼ+error Model 2

(Eᵢⱼ is the expression value of the i^{th} gene in the j^{th} sample)

(Tⱼ is the tissue type of the j^{th} sample)

(Nⱼ is the state of the j^{th} sample (Nⱼ=0 for normal, 1 otherwise))

The model fitting yields, for each gene, a p-value for the T factor, the N factor, and the T*N factor, as well as a sum of squares attributable to each of these factors. Adding the three sums of squares gives the model sum of squares. The R² value is then the ratio of the model sum of squares to the total sum of squares

$${\sum }_{j}^{} {( {E}_{ij} - {\overline{E}}_{i} )}^{2} .$$

TABLE 1

| GLGC | Fragment | Present | Mean Average |
|---|---|---|---|
| Identifier | Name | Frequency | Differential |
| 102271 | AA012709_at | 0.9282 | 190.551 |
| 77300 | AF029357cds_at | 0.9848 | 119.409 |
| 77332 | AF034900mRNA_i_at | 0.989 | 203.019 |
| 77517 | AF081148_s_at | 0.9146 | 52.382 |
| 77576 | AF091561_at | 0.9609 | 62.252 |
| 77615 | AF095927_at | 0.9521 | 40.406 |
| 77721 | AJ132230_g_at | 0.7605 | 62.179 |
| 77738 | D01046_at | 0.8189 | 70.892 |
| 77745 | D10587_at | 0.8261 | 103.633 |
| 80151 | D87840_at | 0.9734 | 83.52 |
| 78209 | M13100cds#1_g_at | 0.9657 | 192.653 |
| 78211 | M13100cds#3_f_at | 0.9867 | 265.171 |
| 78212 | M13100cds#4_f_at | 0.9918 | 128.404 |
| 78213 | M13100cds#5_s_at | 0.9717 | 179.794 |
| 78214 | M13100cds#6_f_at | 0.9817 | 338.825 |
| 78215 | M13101cds_f_at | 0.9256 | 195.555 |
| 81802 | M25584_at | 0.7688 | 108.344 |
| 76571 | M27467_at | 0.8166 | 64.614 |
| 76597 | M74439mRNA_i_at | 0.9709 | 85.002 |
| 76604 | M76767_s_at | 0.9227 | 148.154 |
| 81918 | M83680_at | 0.9692 | 151.235 |
| 84412 | rc_AA799406_at | 0.9722 | 150.886 |
| 84486 | rc_AA799551_g_at | 0.7849 | 110.294 |
| 84567 | rc_AA799745_at | 0.8588 | 123.746 |
| 84748 | rc_AA800684_at | 0.8148 | 47.537 |
| 84809 | rc_AA800881_at | 0.8955 | 98.88 |
| 84830 | rc_AA801017_at | 0.8557 | 56.038 |
| 84832 | rc_AA801025_g_at | 0.9197 | 88.845 |
| 84841 | rc_AA801181_at | 0.8566 | 101.242 |
| 84851 | rc_AA801228_g_at | 0.9251 | 113.4 |
| 84854 | rc_AA801231_at | 0.8871 | 222.933 |
| 99702 | rc_AA818590_at | 0.7573 | 32.931 |
| 98583 | rc_AA819268_at | 0.9357 | 347.913 |
| 100600 | rc_AA819664_at | 0.9852 | 320.9 |
| 84964 | rc_AA848965_at | 0.8342 | 64.375 |
| 85024 | rc_AA849525_i_at | 0.8484 | 45.264 |
| 85060 | rc_AA849730_at | 0.8953 | 66.225 |
| 85158 | rc_AA850117_at | 0.9611 | 228.531 |
| 85262 | rc_AA850595_at | 0.9132 | 86.758 |
| 85466 | rc_AA851405_at | 0.9773 | 114.684 |
| 85474 | rc_AA851439_at | 0.962 | 229.271 |
| 85553 | rc_AA851892_at | 0.9836 | 218.25 |
| 102013 | rc_AA858480_at | 0.8612 | 110.441 |
| 101949 | rc_AA859201_at | 0.9978 | 275.683 |
| 81000 | rc_AA859702_at | 0.8713 | 26.883 |
| 83140 | rc_AA859750_at | 0.7544 | 51.105 |
| 83979 | rc_AA892504_at | 0.82 | 109.04 |
| 81044 | rc_AA892895_r_at | 0.9972 | 499.824 |
| 84111 | rc_AA892959_at | 0.8275 | 37.656 |
| 84145 | rc_AA893127_at | 0.7778 | 96.525 |
| 84310 | rc_AA893980_at | 0.8572 | 69.74 |
| 84392 | rc_AA894340_at | 0.8296 | 31.49 |
| 85633 | rc_AA899265_at | 0.8552 | 56.148 |
| 85635 | rc_AA899278_at | 0.8469 | 56.079 |
| 85698 | rc_AA899664_at | 0.9944 | 414.896 |
| 85712 | rc_AA899723_at | 0.9147 | 112.458 |
| 85771 | rc_AA899991_at | 0.8249 | 124.576 |
| 85831 | rc_AA900348_s_at | 0.9502 | 212.75 |
| 85846 | rc_AA900422_at | 0.9604 | 404.271 |
| 85949 | rc_AA900926_at | 0.8398 | 71.065 |
| 86913 | rc_AA901272_f_at | 0.7765 | 48.604 |
| 87063 | rc_AA924396_at | 0.9271 | 83.43 |
| 76263 | rc_AA924542_s_at | 0.9604 | 62.91 |
| 87182 | rc_AA924830_at | 0.7985 | 40.337 |
| 87211 | rc_AA924964_at | 0.794 | 393.025 |
| 87348 | rc_AA925432_at | 0.9735 | 225.799 |
| 87443 | rc_AA925854_at | 0.8516 | 92.302 |
| 86025 | rc_AA942964_at | 0.9328 | 494.302 |
| 86074 | rc_AA943120_at | 0.855 | 233.325 |
| 86169 | rc_AA943553_g_at | 0.9966 | 665.561 |
| 86209 | rc_AA943738_g_at | 0.9859 | 137.092 |
| 86243 | rc_AA943835_at | 0.7664 | 165.778 |
| 86314 | rc_AA944239_at | 0.949 | 216.561 |
| 86524 | rc_AA945099_g_at | 0.8554 | 54.104 |
| 86629 | rc_AA945805_at | 0.8566 | 68.783 |
| 86724 | rc_AA946166_at | 0.9215 | 75.825 |
| 86727 | rc_AA946181_at | 0.8695 | 169.878 |
| 86837 | rc_AA946499_at | 0.8446 | 63.922 |
| 86846 | rc_AA946528_at | 0.9054 | 279.156 |
| 87736 | rc_AA955911_at | 0.7623 | 70.604 |
| 87993 | rc_AA957063_at | 0.9941 | 391.775 |
| 88267 | rc_AA963170_at | 0.987 | 118.572 |
| 88591 | rc_AA964611_at | 0.9243 | 128.413 |
| 88723 | rc_AA965110_at | 0.7869 | 67.276 |
| 88766 | rc_AA996405_at | 0.8167 | 72.635 |
| 88839 | rc_AA996701_f_at | 0.7552 | 43.716 |
| 89007 | rc_AA997745_at | 0.7736 | 45.566 |
| 89217 | rc_AA997960_at | 0.8546 | 77.485 |
| 89360 | rc_AA998471_i_at | 0.9129 | 284.784 |
| 89468 | rc_AA999041_at | 0.9482 | 133.563 |
| 89701 | rc_AI008674_at | 0.8997 | 100.377 |
| 76186 | rc_AI009141_at | 0.811 | 67.18 |
| 90399 | rc_AI011949_at | 0.7884 | 74.517 |
| 90427 | rc_AI012073_at | 0.7986 | 34.14 |
| 90437 | rc_AI012103_at | 0.7764 | 479.806 |
| 90744 | rc_AI013204_at | 0.9984 | 974.703 |
| 90764 | rc_AI013310_at | 0.7918 | 76.764 |
| 81319 | rc_AI014135_g_at | 0.8066 | 111.16 |
| 91024 | rc_AI029274_at | 0.8263 | 59.624 |
| 81335 | rc_AI029805_at | 0.8404 | 27.604 |
| 91371 | rc_AI030564_at | 0.7837 | 286.222 |
| 91449 | rc_AI030813_at | 0.7509 | 52.319 |
| 91867 | rc_AI044239_i_at | 0.8506 | 43.725 |
| 92024 | rc_AI044638_at | 0.9104 | 212.046 |
| 92444 | rc_AI045686_at | 0.7798 | 72.274 |
| 92887 | rc_AI059209_at | 0.775 | 148.062 |
| 92926 | rc_AI059305_at | 0.9861 | 219.211 |
| 93077 | rc_AI059664_at | 0.9072 | 154.307 |
| 93103 | rc_AI059728_f_at | 0.8303 | 281.846 |
| 93147 | rc_AI059883_at | 0.8219 | 61.436 |
| 93198 | rc_AI060012_at | 0.7549 | 128.285 |
| 93390 | rc_AI069980_at | 0.7936 | 325.454 |
| 93698 | rc_AI070712_at | 0.9272 | 121.653 |
| 93822 | rc_AI071114_at | 0.9722 | 94.206 |
| 93870 | rc_AI071210_at | 0.8462 | 85.695 |
| 93887 | rc_AI071243_at | 0.9775 | 164.564 |
| 93927 | rc_AI071332_at | 0.8399 | 160.424 |
| 93955 | rc_AI071418_at | 0.7542 | 35.773 |
| 94022 | rc_AI071563_at | 0.7516 | 42.418 |
| 94095 | rc_AI071696_f_at | 0.8824 | 255.85 |
| 94127 | rc_AI071763_at | 0.7685 | 27.537 |
| 94183 | rc_AJ071902_at | 0.8004 | 29.416 |
| 93354 | rc_AI071920_at | 0.8101 | 41.866 |
| 94624 | rc_AI073001_at | 0.7888 | 46.337 |
| 94667 | rc_AI073105_at | 0.8006 | 41.572 |
| 94674 | rc_AI073118_at | 0.9816 | 132.82 |
| 94690 | rc_AI073191_at | 0.9111 | 51.687 |
| 96075 | rc_AI101659_at | 0.9988 | 627.052 |
| 96344 | rc_AI102991_at | 0.998 | 389.649 |
| 96381 | rc_AI103202_at | 0.8064 | 149.589 |
| 96436 | rc_AI103415_at | 0.8165 | 44.836 |
| 94805 | rc_AI111950_at | 0.941 | 117.798 |
| 81430 | rc_AI112391_s_at | 0.9029 | 56.828 |
| 95309 | rc_AI144587_at | 0.8708 | 39.214 |
| 95480 | rc_AI145609_at | 0.9806 | 84.399 |
| 81469 | rc_AI146195_at | 0.8938 | 51.357 |
| 95868 | rc_AI169293_at | 0.9127 | 64.184 |
| 96814 | rc_AI169595_at | 0.9206 | 124.878 |
| 96999 | rc_AI170628_at | 0.8098 | 39.401 |
| 97024 | rc_AI170715_at | 0.7835 | 50.309 |
| 97099 | rc_AI170992_at | 0.8404 | 82.011 |
| 97125 | rc_AI171172_i_at | 0.9942 | 137.021 |
| 97394 | rc_AI172069_at | 0.9579 | 55.272 |
| 97458 | rc_AI172218_at | 0.9678 | 136.643 |
| 97601 | rc_AI172576_at | 0.8256 | 38.281 |
| 97690 | rc_AI175266_at | 0.9973 | 335.31 |
| 97837 | rc_AI175830_at | 0.7816 | 27.925 |
| 97962 | rc_AI176309_at | 0.9542 | 86.007 |
| 98068 | rc_AI176625_at | 0.8551 | 152.373 |
| 98219 | rc_AI177089_at | 0.7707 | 28.18 |
| 98232 | rc_AI177117_at | 0.7661 | 54.616 |
| 98277 | rc_AI177251_at | 0.8129 | 49.094 |
| 98367 | rc_AI177595_at | 0.8043 | 52.792 |
| 98370 | rc_AI177603_at | 0.798 | 37.734 |
| 98563 | rc_AI178446_at | 0.8241 | 98.564 |
| 98796 | rc_AI179239_at | 0.992 | 158.966 |
| 98850 | rc_AI179411_at | 0.9052 | 78.786 |
| 99019 | rc_AI180081_at | 0.9738 | 389.838 |
| 99327 | rc_AI228249_at | 0.9917 | 429.5 |
| 99339 | rc_AI228279_at | 0.8721 | 81.722 |
| 99439 | rc_AI228722_at | 0.8644 | 49.792 |
| 99810 | rc_AI230308_at | 0.9803 | 180.54 |
| 99878 | rc_AI230562_at | 0.9277 | 84.362 |
| 81702 | rc_AI230572_at | 0.8913 | 58.278 |
| 100117 | rc_AI231330_at | 0.751 | 40.863 |
| 100183 | rc_AI231565_at | 0.9039 | 104.091 |
| 100394 | rc_AI232347_at | 0.8852 | 120.621 |
| 100501 | rc_AI232722_at | 0.8026 | 180.831 |
| 100698 | rc_AI233529_f_at | 0.8144 | 72.074 |
| 100818 | rc_AI233965_at | 0.9171 | 60.938 |
| 100819 | rc_AI233966_at | 0.8467 | 142.163 |
| 101057 | rc_AI235032_at | 0.9552 | 125.501 |
| 101104 | rc_AI235232_at | 0.8299 | 102.496 |
| 101115 | rc_AI235272_at | 0.7574 | 35.891 |
| 101135 | rc_AI235315_at | 0.7708 | 60.792 |
| 101275 | rc_AI235821_f_at | 0.7721 | 181.906 |
| 101388 | rc_AI236169_at | 0.9237 | 82.826 |
| 101477 | rc_AI236475_at | 0.8718 | 156.175 |
| 101721 | rc_AI237366_at | 0.9603 | 63.197 |
| 80595 | rc_AI639114_at | 0.8775 | 21.093 |
| 80849 | rc_AI639391_at | 0.7655 | 61.047 |
| 80925 | rc_AI639465_f_at | 0.9602 | 142.244 |
| 83528 | rc_H31217_at | 0.7871 | 28.269 |
| 83544 | rc_H31535_at | 0.8248 | 95.236 |
| 78445 | S50461_s_at | 0.7606 | 35.999 |
| 78545 | S70803_at | 0.884 | 93.026 |
| 78574 | S74572_g_at | 0.791 | 32.907 |
| 78678 | S90449_at | 0.8728 | 27.837 |
| 82688 | U37138_at | 0.8904 | 47.73 |
| 82488 | U49099_at | 0.9579 | 89.613 |
| 76764 | U61184_at | 0.8679 | 32.322 |
| 78926 | U87971_g_at | 0.8219 | 29.276 |
| 78969 | X05472cds#1_s_at | 0.923 | 129.01 |
| 78971 | X05472cds#3_f_at | 0.8638 | 129.503 |
| 79009 | X13527cds_s_at | 0.7644 | 118.765 |
| 79081 | X53581cds#3_f_at | 0.908 | 166.237 |
| 79840 | X53944_at | 0.9981 | 196.006 |
| 79230 | X89697cds_at | 0.806 | 34.392 |

### Example 2

### Quantitative PCR Analysis of Expression Levels Using the Control Genes

The expression levels of one or more genes listed in Table 1 may be used to normalize gene expression data produced using quantitative PCR analysis. For example, the sequences may be used as Taqman probes, along with the forward and reverse primers for a gene in Table 1. Real time PCR detection may be accomplished by the use of the ABI PRISM 7700 Sequence Detection System. The 7700 measures the fluorescence intensity of the sample each cycle and is able to detect the presence of specific amplicons within the PCR reaction. The TaqMan® assay provided by Perkin Elmer may be used to assay quantities of RNA. The primers may be designed from each of the genes identified in Table 1 using Primer Express, a program developed by PE to efficiently find primers and probes for specific sequences. These primers may be used in conjunction with SYBR green (Molecular Probes), a nonspecific double-stranded DNA dye, to measure the expression level mRNA corresponding to the expression levels of each gene. This gene expression data may then be used to normalize gene expression data of other test genes.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

We claim:

1. A method of identifying at least one gene that is consistently expressed across different cell or tissue types in an organism, comprising:
(a) preparing gene expression profiles for different cell or tissue types from the organism;
(b) calculating the percent variability of expression using a one-factor or two-factor ANOVA analysis for at least one gene in each of the profiles across the different cell or tissue types; and
(c) selecting any gene whose percent variability of expression indicates that the gene is consistently expressed across the different cell or tissue types.

2. A method of claim 1, wherein the R² value from the one-factor or two-factor ANOVA analysis is a measure of percent variability of expression for the at least one gene.

3. A method of claim 2, wherein the R² value from the one-factor or two-factor ANOVA analysis is less than or equal to about 12.

4. A method of claim 1, wherein the different cell or tissue types comprise greater than about 10 different cell or tissue types.

5. A method of claim 1, wherein the different cell or tissue types comprise greater than about 25 different cell or tissue types.

6. A method of claim 1, wherein the different cell or tissue types comprise greater than about 50 different cell or tissue types.

7. A method of claim 4, wherein the cell or tissue types comprise normal and diseased cell or tissue types.

8. A method of claim 1, wherein the organism is a mammal.

9. A method of claim 8, wherein the mammal is a rat.

10. A method of claim 1, wherein the expression profiles are generated by querying a gene expression database for the expression level of at least one gene in different cell or tissue types from the organism or from a cell line.

11. A set of probes comprising at least two probes that specifically hybridize to a gene identified by the method of claim 1.

12. A set of probes according to claim 11, wherein the set comprises probes that specifically hybridize to at least about 10 genes.

13. A set of probes according to claim 11, wherein the set comprises probes that specifically hybridize to at least about 25 genes.

14. A set of probes according to claim 11, wherein the set comprises probes that specifically hybridize to at least about 50 genes.

15. A set of probes according to claim 11, wherein the set comprises probes that specifically hybridize to at least about 100 genes.

16. A set of probes according to claim 11, wherein the probes are attached to a single solid substrate.

17. A set of probes of claim 16, wherein the solid substrate is a chip.

18. A method of normalizing the data from a nucleic acid detection assay comprising:
(a) detecting the expression level for at least one gene in a nucleic acid sample; and
(b) normalizing the expression of said at least one gene with the detected expression of an control gene identified by the method of claim 1.

19. A method of claim 18, wherein step (b) comprises normalizing the expression level of said at least one gene with the expression levels of at least about 10 control genes.

20. A method of claim 18, wherein step (b) comprises normalizing the expression level of said at least one gene with the expression levels of at least about 25 control genes.

21. A method of claim 18, wherein step (b) comprises normalizing the expression level of said at least one gene with the expression levels of at least about 50 control genes.

22. A method of claim 18, wherein step (b) comprises normalizing the expression level of said at least one gene with the expression levels of at least about 100 control genes.

23. A method of claim 18, wherein the assay is quantitative.

24. A method of claim 18, wherein the assay is a hybridization reaction conducted on a solid substrate.

25. A method of claim 24, wherein the solid substrate is an oligonucleotide array.

26. A method of claim 25, wherein the array comprises oligonucleotide probes that are complementary to the control genes.

27. A method of claim 18, wherein the assay is a polymerase chain reaction.

28. A set of probes comprising at least two probes that specifically hybridize to a gene of Table 1 or a gene exhibiting about 95% nucleotide sequence identity to a gene of Table 1.

29. A set of probes of claim 28, comprising probes that specifically hybridize to at least about 10 genes of Table 1.

30. A set of probes of claim 28, comprising probes that specifically hybridize to at least about 25 genes of Table 1.

31. A set of probes of claim 28, comprising probes that specifically hybridize to at least about 50 genes of Table 1.

32. A set of probes of claim 28, comprising probes that specifically hybridize to at least about 100 genes of Table 1.

33. A set of probes of claim 28, wherein the probes are attached to a single solid substrate.

34. A set of probes of claim 33, wherein the solid substrate is a chip.

35. A method of normalizing the data from a nucleic acid detection assay comprising:
(a) detecting the expression level for at least one gene in a nucleic acid sample; and
(b) normalizing the expression of said at least one gene with the detected expression of a control gene of Table 1.

36. A method of claim 35, wherein step (b) comprises normalizing the expression level of said at least one gene with the expression levels of at least about 10 control genes of Table 1.

37. A method of claim 35, wherein step (b) comprises normalizing the expression level of said at least one gene with the expression levels of at least about 25 control genes of Table 1.

38. A method of claim 35, wherein step (b) comprises normalizing the expression level of said at least one gene with the expression levels of at least about 50 control genes of Table 1.

39. A method of claim 35, wherein step (b) comprises normalizing the expression level of said at least one gene with the expression levels of at least about 100 control genes of Table 1.

40. A method of claim 35, wherein the assay is quantitative.

41. A method of claim 35, wherein the assay is a hybridization reaction conducted on a solid substrate.

42. A method of claim 41, wherein the solid substrate is an oligonucleotide array.

43. A method of claim 42, wherein the array comprises oligonucleotide probes that are complementary to the control genes.

44. A method of claim 35, wherein the assay is a polymerase chain reaction.

45. A method of claim 18, wherein the normalizing of step (b) comprises dividing the expression level for said at least one gene by the detected expression level of said control gene.

46. A method of identifying at least one gene that is consistently expressed across different cell or tissue types in an organism or cell line, comprising:
(a) querying a gene expression database for the expression level of at least one gene in different cell or tissue types from the organism or cell lines;
(b) calculating the percent variability of expression using a one-factor or two-factor ANOVA analysis for said at least one gene across the different cell or tissue types or cell lines; and
(c) identifying at least one gene whose percent variability of expression indicates that the gene is consistently expressed across the different cell or tissue types or cell lines.

47. A method of claim 46, wherein the R² value from the one-factor or two-factor ANOVA analysis is a measure of percent variability of expression for the at least one gene.

48. A method of claim 47, wherein the R² value from the one-factor or two-factor ANOVA analysis is less than or equal to about 12.

49. A method of claim 46, wherein the different cell or tissue types comprise greater than about 10 different cell or tissue types.

50. A method of claim 46, wherein the different cell or tissue types comprise greater than about 25 different cell or tissue types.

51. A method of claim 46, wherein the different cell or tissue types comprise greater than about 50 different cell or tissue types.

52. A method of claim 46, wherein the cell or tissue types comprise normal and diseased cell or tissue types.

53. A method of claim 46, wherein the organism is a mammal.

54. A method of claim 54, wherein the mammal is a rat.

55. A method of identifying a nucleic acid molecule whose level of expression is invariant across two or more cell or tissue samples, comprising:
(a) determining the variation in the expression level of the nucleic acid molecule (R² value) from two or more cell or tissue samples by one factor or two factor analysis of variation (ANOVA);
(b) comparing the R² value for the nucleic acid molecule to a threshold value, wherein the expression level of the nucleic acid molecule is considered to be invariant if the R² value is less than the threshold value; and
(c) identifying a nucleic acid molecule whose level of expression is invariant across two or more cell or tissue samples.

56. A method of normalizing data from a nucleic acid detection assay comprising:
(a) detecting the expression level for at least one gene in a nucleic acid sample; and
(b) normalizing the expression level of said at least one gene with the detected expression level of an invariant gene identified by the method of claim 55.
